(19) United States
(12) Patent Application Publication
(10) Pub. No.: US 2018/0169021 A1
(43) Pub. Date: Jun. 21, 2018

(54) RANOLAZINE MULTIPLE COMPRESSED TABLETS

(71) Applicant: INTERQUIM, S.A., Sant Cugat Del Vallès (ES)

(72) Inventors: Javier URBANO HURTADO, Sant Cugat Del Vallès (ES); Pablo MARTIN SAIZ, Sant Cugat Del Vallès (ES)

(73) Assignee: INTERQUIM, S.A., Sant Cugat Del Vallès (ES)

(21) Appl. No.: 15/737,628

(22) PCT Filed: Jul. 1, 2016
(86) PCT No.: PCT/EP2016/065534

§ 371 Date: Dec. 18, 2017

(30) Foreign Application Priority Data
Jul. 2, 2015 (EP) 15174977.7

(51) Int. Cl. A61K 9/20; A61K 31/495

(52) U.S. Cl. CPC A61K 9/2086; A61K 31/495; A61K 9/2095; A61K 9/2054

(57) ABSTRACT

The present invention is in the field of drug delivery, more specifically in the field of ranolazine delivery, more specifically ranolazine extended release delivery using multiple compressed tablets of ranolazine. The geometry of the tablets allows obtaining different dissolution profiles modifying the size and composition of the different pharmaceutical compositions.

### FILED OF THE INVENTION

The present invention is in the field of drug delivery, more specifically in the field of extended release drug delivery, and deals with ranolazine extended release delivery using multiple compressed tablets.

### BACKGROUND OF THE INVENTION

Ranolazine was first disclosed in EP0126449 A1, has the systematic name N-(2,6-dimethylphenyl)-2-(4-(2-hydroxy-3-(2-methoxyphenoxy)propyl)piperazin-1-yl)acetamide and the following chemical structure:

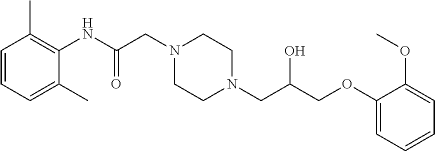 [SMILES: COc1ccccc1OCC(O)CN1CCN(CC(=O)Nc2c(C)cccc2C)CC1]

Ranolazine is marketed as Ranexa® in 375, 500, 750 and 1000 mg extended release tablets as antianginal agent.

The solubility of ranolazine is pH dependent and according to WO03086401 A1 the solubility of ranolazine is as follows:

| Solution pH | Solubility (mg/mL) | USP Solubility Class |
|---|---|---|
| 4.81 | 161 | Freely Soluble |
| 4.89 | 73.8 | Soluble |
| 4.90 | 76.4 | Soluble |
| 5.04 | 49.4 | Soluble |
| 5.35 | 16.7 | Sparingly Soluble |
| 5.82 | 5.48 | Slightly soluble |
| 6.46 | 1.63 | Slightly soluble |
| 6.73 | 0.83 | Very slightly soluble |
| 7.08 | 0.39 | Very slightly soluble |
| 7.59* | 0.24 | Very slightly soluble |
| 7.79 | 0.17 | Very slightly soluble |
| 12.66 | 0.18 | Very slightly soluble |

As shown in the previous table, ranolazine is much more soluble at acidic pH than in neutral or basic pH. The rate at which a 100% ranolazine tablet dissolves at different pH values is shown in FIG. 1, where it is clearly observed that at pH 1 in less than 30 minutes all the ranolazine is dissolved, while at pH 6.8 in 30 minutes only around 63% of the ranolazine is dissolved and in 12 hours ranolazine is not yet fully dissolved.

According to WO0166093 A2: “[o]ne problem with conventional oral dosage formulations is that they are not ideally suited to ranolazine and its pharmaceutically acceptable salts, because the solubility of ranolazine is relatively high at the low pH that occurs in the stomach. Furthermore ranolazine also has a relatively short plasma half-life. The high acid solubility property of ranolazine results in rapid drug absorption and clearance, causing large and undesirable fluctuations in plasma concentration of ranolazine and a short duration of action, thus necessitating frequent oral administration for adequate treatment”. Therefore the preferred method of administration of ranolazine is as an extended release formulation. Thus, several ranolazine extended release formulations have been disclosed based on different technologies.

The use of pH dependent polymers is disclose in WO03099281 A2, CN101637442 A, WO0013686 A2, WO0013687 A2, WO0166093 A2, CN101066253 A, WO11036677 A2, WO12152440 A1 or TR201203341 A2.

The use of acids is disclosed in JP2000336032 A2.

The use of lipid compounds is disclosed in WO10137040 A2 or WO11107750 A2.

The use of pellets coated only with pH independent binders is disclosed in CN102125523 A, CN101066254 A, CN102125523 A or CN103751112 A. CN101176723 A, CN1891218 A, IN02204MU2009 A and WO06074398 A2.

The use of a release mechanism based on the pH of the medium (pH dependent polymers and acids) is convenient because in the digestive system different values of pH can be found: acidic in the stomach and neutral/basic in the bowel. However, the inventors of the present invention have found that, with this kind of release mechanism, ranolazine formulations show a high inter-subject variability due to inter-subject variations in stomach pH and transit time variability, which results in high inter-subject bioavailability variability. This issue can be amplified in conditions such as gastroparesis, hyperchlorhydria or achlorhydria.

Ranexa® marketed composition is based on pH dependent binders. FIG. 2 shows that the dissolution of ranolazine in Ranexa® is heavily dependent on the pH of the medium.

The alternative of using coated granules was found not desirable either, because when coated granules are compressed into tablets the coating may be damaged irregularly and will result in increasing variability between tablets due to the irregularly damaged coating. This can be solved by using capsules, but this is not desirable in the case of high dosage drugs (such as ranolazine, which is administered up to 1000 mg) because, since the capsule content is not compressed, it is very bulky and occupies a large volume; thus requiring large capsules (which are hard to swallow). Not only that, but capsules are also usually difficult to fill accurately and their preparation is lengthier and costlier than that of tablets.

A part form the previously mentioned problems, the preparation of coated granules has many drawbacks, such as variability in granule size and form or coating thickness and the long times required to coat the granules. Since the coating is functional, variability in its thickness may result in increased variability in the release rate depending on the thickness of the coating.

The alternative of using lipid compounds such as fats, oils or waxes to obtain the extended release is not desirable either, because such excipients are difficult to handle due to their viscosity and sticky properties and results in a high variability in the drug content within the manufactured dosage forms. Not only that, but fats, oils or waxes may also have different behaviour when taken with or without food.

Therefore there is a need to provide new ranolazine extended release pharmaceutical compositions which overcome the previous problems including the variability (inter-subject, interaction with food, content uniformity, . . . ) and that can be easily modified to obtain different dissolution profiles in time as desired.

### SUMMARY OF THE INVENTION

One aspect of the invention is a multiple compressed tablet obtainable by a process comprising at least two compression cycles, wherein
in each of the compression cycles a pharmaceutical composition, comprising one or more pharmaceutically acceptable excipients, is used,
at least one of such pharmaceutical compositions comprises one or more release retardant agents,
at least two of such pharmaceutical compositions comprise ranolazine and have a different quantitative and/or qualitative composition.

Another aspect of the invention is a multiple compressed tablet according to the invention for use in the treatment of angina pectoris.

Another aspect of the invention is a process for the preparation of the multiple compressed tablet according to the invention comprising:
a) independently mixing all the components of all the pharmaceutical compositions,
b) optionally tableting one or more pharmaceutical compositions,
c) either
   i. charging and optionally precompressing a pharmaceutical composition different from the one used in the previous cycle on the result of the previous cycle, or
   ii. placing a tablet of step b), prepared using a pharmaceutical composition different from the one used in the previous cycle, on the result of the previous cycle,
d) repeating step c) at least one time,
e) compressing the result of the previous step.

### Definitions

A pharmaceutically acceptable excipient is a component of a pharmaceutical composition or formulation which has one or more functions and is suitable to be administered to any animal including mammals and humans. Some of the functions that the excipient may perform are: release retardant agent, diluent, binder, disintegrant, glidant, lubricant, coating, colorant, flavouring agent, sweetener, and the like.

A release retardant agent is a pharmaceutical acceptable excipient that, when incorporated in a pharmaceutical composition, reduces the rate at which a drug is released from the pharmaceutical composition.

A pH dependent release retardant agent is a release retardant agent that, when incorporated in a pharmaceutical composition, makes the rate at which the drug is released dependent on the pH of the dissolution media.

A pH independent release retardant agent is a release retardant agent that, when incorporated in a pharmaceutical composition, makes the rate at which the drug is released substantially independent of the pH of the dissolution media. Suitable pH independent release retardant agents are pH independent polymers and pH independent binders.

A pH independent polymer is a polymeric pH independent release retardant agent. Examples of pH independent polymers are: hydroxypropyl methylcellulose (also known as hypromellose or HPMC), hydroxypropylcellulose, methylcellulose, polyvinylpyrrolidone (also known as povidone or PVP), neutral poly(meth)acrylate esters and the like.

A diluent is an inert pharmaceutically acceptable excipient that provides bulk to the pharmaceutical composition, facilitates the manufacturing process of the dosage form as well as improves the uniformity of content of the active ingredient in the composition. Suitable examples of diluents are: microcrystalline cellulose, lactose (including but not limited to lactose USP, anhydrous lactose USP and spray-dried lactose USP), starch (including but not limited to maize starch, dry starch and directly compressible starch and hydrolyzed starch (including but not limited to Celutab®)), mannitol, sorbitol, inositol, sucrose-based diluents (including but not limited to sucrose, confectioner's sugar and sugar spheres NF), dextrose (including but not limited to Cerelose® and dextrose monohydrate), dicalcium phosphate (including but not limited to dicalcium phosphate trihydrate), monobasic calcium sulfate monohydrate, calcium sulfate dihydrate, calcium lactate trihydrate (including but not limited to calcium lactate trihydrate granular NF), calcium carbonate, dextrates (e. g., Emdex®), hydrolyzed cereal solids (including but not limited to Matron products and Mor-Rex), amylose, Recel, powdered cellulose (including but not limited to Elcema®), glycine, bentonite and the like.

A binder is a pharmaceutically acceptable excipient that holds the components of a pharmaceutical composition together. Suitable binders are: polyvinylpyrrolidone (preferably a grade with viscosity between 1.5 to 8.5 and more preferable with viscosity 3.5 to 5.5 mPa·s), starch (including but not limited to maize starch and pregelatinized starch), copovidone, gum acacia, gum arabica, gelatine, cellulose and derivatives thereof (including but not limited to cellulose esters, cellulose ethers and hydroxypropylcellulose), xylitol, sorbitol, maltitol, polyethylene glycol and the like.

A disintegrant is a pharmaceutically acceptable excipient that included in solid pharmaceutical forms, such as tablets or granules, facilitate its break up or disintegration in an aqueous environment. Suitable disintegrants are starches (including but not limited to sodium starch glycolate, corn starch, potato starch, maize starch, modified starches and pregelatinized corn starches (including but not limited to National 1551 and National 1550)), crospovidone, clays (including but not limited to bentonite, bentonite magma, purified bentonite, kaolin, ball clay, common clay, magnesium aluminium silicate, magnesium trisilicate and shale, and fire clay), celluloses (including but not limited to purified cellulose, methylcellulose and carmellose sodium (also known as sodium carboxymethylcellulose), cross-linked cellulloses, such as cross-linked carmellose (croscarmellose) and its salts, including sodium croscarmellose), alginates, gums (such as agar, guar, locust bean, karaya, pectin, and tragacanth gums) and the like.

A glidant is a pharmaceutically acceptable excipient that eases powder flow of pharmaceutical mixtures. Suitable glidants are anhydrous colloidal silica (a.k.a. silicon dioxide), talc, magnesium carbonate, glyceryl behenate (including but not limited to Compritol® 888), hydrogenated vegetable oils (including but not limited to Sterotex®), waxes, boric acid, sodium benzoate, sodium acetate, sodium chloride, DL-leucine, polyethylene glycols (including but not limited to Carbowax® 4000 and Carbowax® 6000), sodium oleate and the like.

A lubricant is pharmaceutical excipient that prevents the ingredients from sticking to the tablet dies. Suitable lubricants are sodium stearyl fumarate, stearic acid, magnesium stearate, calcium stearate, magnesium lauryl sulfate, talc, silica, and the like.

A coating is defined here as a layer of a given thickness that surrounds the entire tablet surface.

A film coating is a thin layer (of about 0.02-0.5 mm) that surrounds a dosage form, here a tablet. The coating may perform different functions: aesthetic, ease the swallowing, modify the release of the drug (e.g. enteric coating, sustained release, . . . ). Suitable coatings are coatings based on HPMC, poly(vinyl alcohol). Commercial examples are Opadry®, Opadry® 200, Opadry® amb II, Opadry® Fx™, Opadry® II, Opalux®.

Sugar coating involves the deposition from an aqueous solution of coatings based on sugars, typically sucrose.

A colorant is a product that provides colour to the pharmaceutical composition. Suitable colorants are: C.I. Pigment White 6, C.I. Natural Brown 10, C.I. Food Red 12, C.I. Food Red 17, C.I. Food Red 9, C.I. Food Red 3, C.I. Food Orange 8, C.I. Natural Red 4, C.I. Red 87, C.I. Food Red 14, C.I. Pigment Red 101 & 102, C.I. Food Red 7, C.I. Food Red 10, C.I. Food Orange 5, C.I. Food Orange 6, C.I. Natural Yellow 3, C.I. Food Yellow 13, C.I. Pigment Yellow 42 & 43, C.I. Food Yellow 13, C.I. Food Yellow 3, C.I. Food Yellow 4, C.I. Natural Green 3, C.I. Natural Green 3, C.I. Food Green 3, C.I. Food Green 4, C.I. Food Blue 2, C.I. Food Blue 1, C.I. Food Blue 5, C.I. Food Black 1, C.I. Pigment Black 11, C.I. Food Black 3 and the like.

A flavouring agent is a product that provides flavour to the pharmaceutical composition. Suitable flavouring agents are strawberry flavour, cherry flavour, banana flavour, mint flavour, orange, lemon, vanillin, peppermint, grape and the like.

A sweetener is an excipient that provides sweet taste to the pharmaceutical composition. Suitable sweeteners are preferably selected from the group consisting of sugars (such as sucrose, fructose, glucose and the like), artificial sweeteners (such as saccharin or its pharmaceutically acceptable salts (such as saccharin sodium), cyclamate or its pharmaceutically acceptable salts (such as cyclamate sodium), aspartame, acesulphame or its pharmaceutically acceptable salts (such as acesulphame potassium), sucralose, neohespiridin dihydrochalcone, naringin dihydrochalcone and the like), and mixtures thereof.

Further suitable excipients and its role can be found on Handbook of Pharmaceutical Excipients, APhA Publications 5^{th} edition 2005, edited by Raymond C. Rowe, Paul J. Sheskey, Sian C. Owen, ISBN-10: 1582120587. In this reference synonyms of the excipients cited here and further examples of specific types of excipients discussed here can be found.

pH values lower than 7 mean acidic medium, pH=7 is neutral and pH values higher than 7 mean basic medium.

According to Remington: The Science and Practice of Pharmacy, 21^{st} edition, 2006, page 890, multiple compressed tablets are compressed tablets made by more than one compression cycle. This definition includes layered tablets (bilayer tablets, trilayer tablets and, more generally, multilayer tablets) as well as press-coated tablets and inlay tablet.

A multilayer tablet is a tablet prepared by at least precompressing one or more additional pharmaceutical compositions on a previously at least precompressed pharmaceutical composition.

A bilayer tablet is a multilayer tablet which is prepared with only one additional pharmaceutical composition and resulting in a tablet with two layers.

A trilayer tablet is a multilayer tablet which is prepared with one or two additional pharmaceutical compositions and resulting in a tablet with three layers. Only one additional pharmaceutical composition is required if after forming a bilayer tablet, the same composition of the first layer is used.

A press-coated tablet is a tablet prepared by compressing a pharmaceutical composition around a previously formed tablet. The press-coated tablets are also referred as dry coated tablet, tablet-into-tablet or tablet-within-a-tablet. Alternatively, the previous form tablet can be prepared by hot melt extrusion or any other method that allows preparing a solid compact pharmaceutical form.

An inlay tablet is a tablet prepared by compressing a pharmaceutical composition around a previously formed tablet, but wherein the inner tablet is not completely surrounded by this pharmaceutical composition and thus one of the surfaces of the inner tablet is exposed. Alternatively, the previous form tablet can be prepared by hot melt extrusion or any other method that allows preparing a solid compact pharmaceutical form.

A compression cycle in the context of the present invention is a step that includes the compression or precompression of a pharmaceutical composition.

Precompressing is applying force to a mixture, being that force lower than that required for compressing a mixture into a tablet. Typically, the force applied in precompression ranges from 0.2 kN to 5.0 kN, most commonly from 0.5 kN to 2 kN.

Compressing is applying enough force to a mixture to compress that mixture into a tablet. This is sometimes referred as tabletting. Typically, the force applied in compression ranges from 5 kN to 100 kN, most commonly from 5 kN to 75 kN and most commonly, from kN to 30 kN.

A drug is a chemical substance used in the treatment, cure, prevention, or diagnosis of disease or used to otherwise enhance physical or mental well-being of a mammal including humans and when present in a tablet is in an amount enough to produce such effect.

Outer pharmaceutical compositions are the pharmaceutical compositions present in the trilayer, multilayer, press-coated or inlay tablets which contain the point further away from the geometrical centre of the tablet.

Inner pharmaceutical compositions are the pharmaceutical compositions present in the press-coated or inlay tablets which are totally or partially surrounded by the outer pharmaceutical compositions.

Intermediate pharmaceutical compositions in the trilayer and multilayer tablets are the pharmaceutical compositions found between the two outer pharmaceutical compositions.

Charging a pharmaceutical composition in a die means placing the powder or granules in the die prior to precompression or compression.

Unless otherwise stated, aqueous dissolution media is deionized water wherein, optionally, the pH is adjusted as described or as known in the art.

### DESCRIPTION OF FIGURES

FIG. 1 shows the % of dissolution in time of 500 mg ranolazine compressed into tablets in 900 mL of two aqueous dissolution media at pH values of 1.0 (0.1 M HCl) and 6.8 (0.05 M KH₂PO₄/Na₂HPO₄ buffer adjusting to the desired pH using 85% H₃PO₄ or 2.0 M NaOH) using paddle stirred at 100 rpm.

FIG. 2 shows the dissolution of 1000 mg Ranexa® tablets at pH values of 1.0, 4.5 and 6.8. The 1000 mg Ranexa® tablets are dissolved in 900 mL of aqueous dissolution media using paddle stirring at 50 rpm. The pH 1.0 is obtained using 0.1 M HCl. The pH 4.5 is obtained using 0.2 M acetic acid sodium acetate buffer and adjusting to the desired pH using glacial acetic acid or 2.0 M NaOH. The pH 6.8 is obtained using 0.05 M KH₂PO₄/Na₂HPO₄ buffer and adjusting to the desired pH using 85% H₃PO₄ or 2.0 M NaOH.

FIG. 3 shows cross-sectional views of representative kinds of tablets. The drawings are based on round tablets which have flat surfaces. The different pharmaceutical composition of the different layers or regions is shown graphically. The invention is not in any way limited to round tablets which have flat surfaces.

FIG. 3A shows a tablet with two layers, each prepared using different pharmaceutical compositions. This is commonly known as a bilayer tablet.

FIG. 3B shows a tablet with three layers, the outer and inner fractions are prepared using different pharmaceutical compositions. The two outer pharmaceutical compositions can be the same or different. This is commonly known as a trilayer tablet.

FIG. 3C shows a tablet which is made of an inner tablet fully surrounded by an outer pharmaceutical composition, wherein the outer and the inner fractions have a different pharmaceutical composition. This is commonly known as a press-coated tablet.

FIG. 3D shows a tablet surrounded by another pharmaceutical composition on one base and on the entire lateral surface, wherein the other base is exposed to the dissolution media. This is commonly known as an inlay tablet.

FIG. 3E shows a tablet made of different layers of different pharmaceutical compositions. Some or all of the non-touching layers may have the same pharmaceutical composition as shown in the figure or not. This is commonly known as a multilayer tablet.

FIG. 4 shows different shapes of tablet bases.

FIG. 4A represents a round tablet.

FIG. 4B represents an oblong tablet.

FIG. 4C represents an oval tablet.

FIG. 4D represents a square tablet.

FIG. 4E represents a rectangle tablet.

FIG. 4F represents a diamond tablet.

FIG. 4G represents a 3 sided tablet.

FIG. 4H represents a 5 sided tablet.

FIG. 4I represents a 6 sided tablet.

FIG. 4J represents a 7 sided tablet

FIG. 4K represents an 8 sided tablet.

FIGS. 5A to 5M show the dissolution profile of several example compositions using the following dissolution method: the tablets to be measured are placed in a vessel with 800 mL of 0.1 M HCl aqueous solution stirred with paddles at 100 rpm, the concentration of ranolazine in the aqueous solution is measured at different times until 1 h. Afterwards 100 mL of 0.51 M Na₃PO₄ solution are added and the pH is adjusted to pH 6.8 using 2.0 M HCl or 2.0 M NaOH. The samples are taken at different times and the ranolazine content is measured using the area obtained with a HPLC apparatus with UV detector and compared to a reference.

The reference is prepared by dissolving the full amount of ranolazine present in the tablet to be measured in the same amount of aqueous dissolution media of the sample. If it is desired, the reference can be prepared by using the same ranolazine to aqueous dissolution media ratio found in the measurement vessel.

FIG. 5A shows the dissolution profile of the composition obtained in Example 5.

FIG. 5B shows the dissolution profile of the composition obtained in Example 6.

FIG. 5C shows the dissolution profile of the composition obtained in Example 7.

FIG. 5D shows the dissolution profile of the composition obtained in Example 8.

FIG. 5E shows the dissolution profile of the composition obtained in Example 16.

FIG. 5F shows the dissolution profile of the composition obtained in Example 17.

FIG. 5G shows the dissolution profile of the composition obtained in Example 22.

FIG. 5H shows the dissolution profile of the composition obtained in Example 24.

FIG. 5I shows the dissolution profile of the composition obtained in Example 25.

FIG. 5J shows the dissolution profile of the composition obtained in Example 29.

FIG. 5K shows the dissolution profile of the composition obtained in Example 30.

FIG. 5L shows the dissolution profile of the marketed 1000 mg Ranexa® tablets.

FIG. 5M is the combination of FIGS. 5A to 5L.

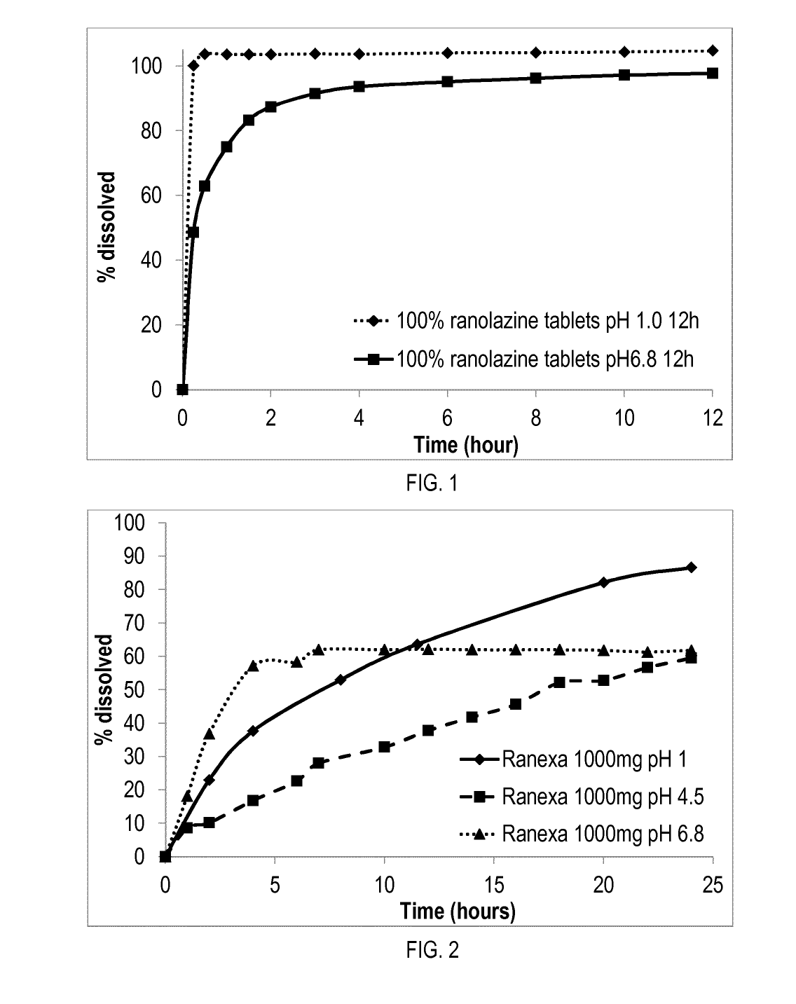

### DETAILED DESCRIPTION OF THE INVENTION

The inventors have found that the present invention allows preparing extended release tablets showing different ranolazine release rates thanks to the possible modulation by means of the relative amount of ranolazine and the content of release retardant agent in each of the pharmaceutical compositions used in the different compression cycles. For instance, when the fraction with more release retardant agent represents a larger portion of the tablet, greater extended release effect is found.

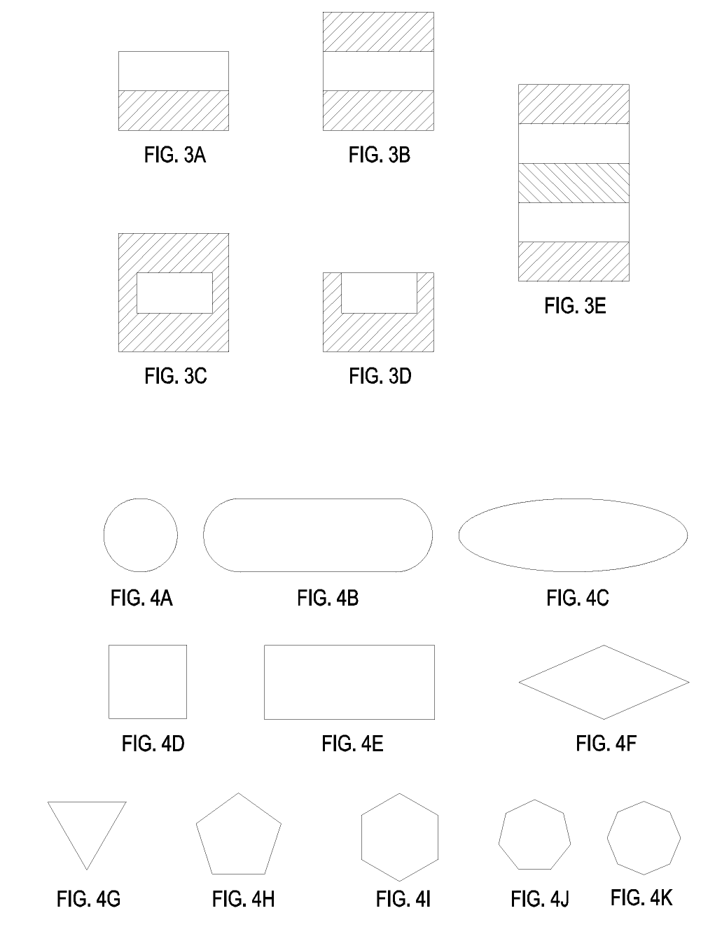

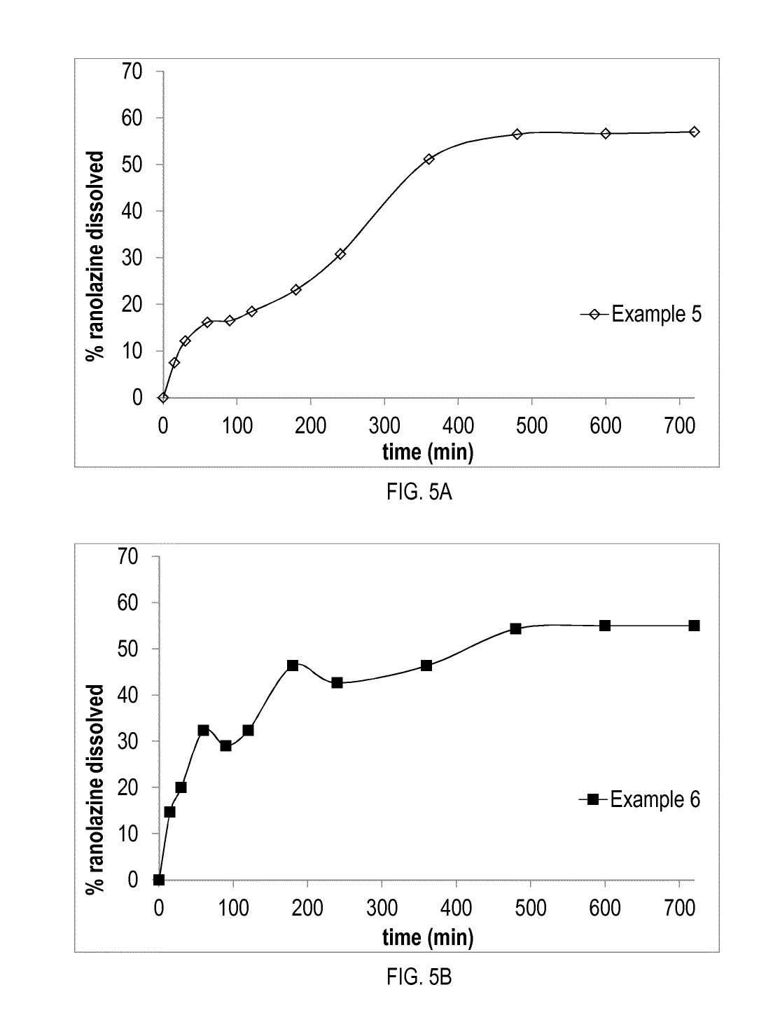

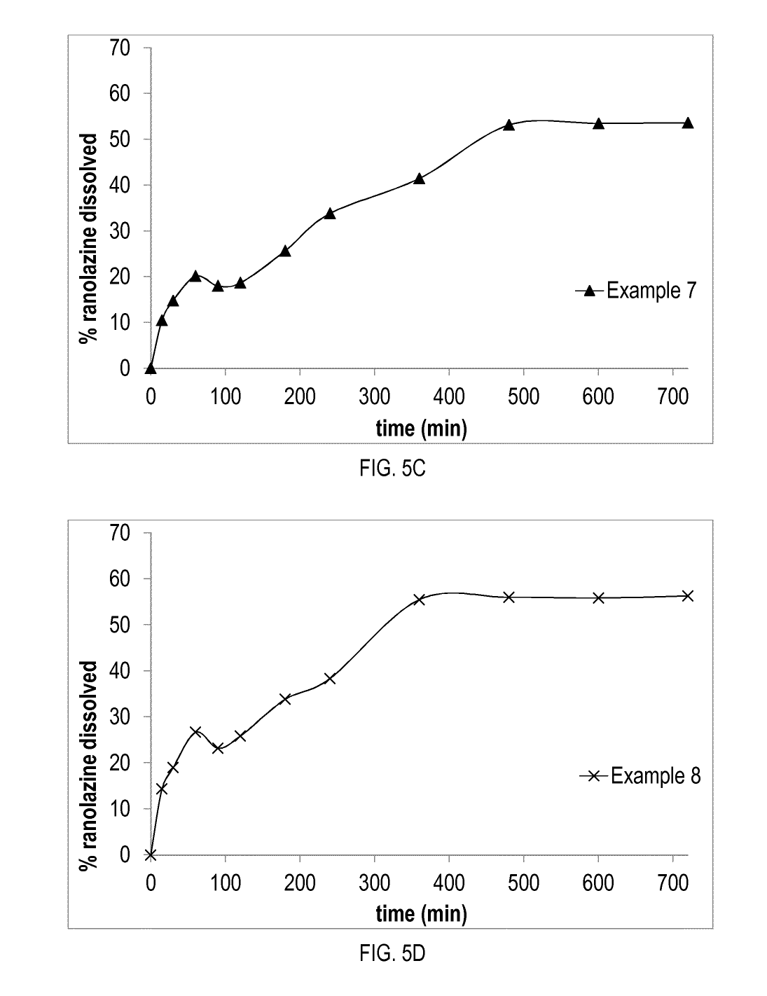

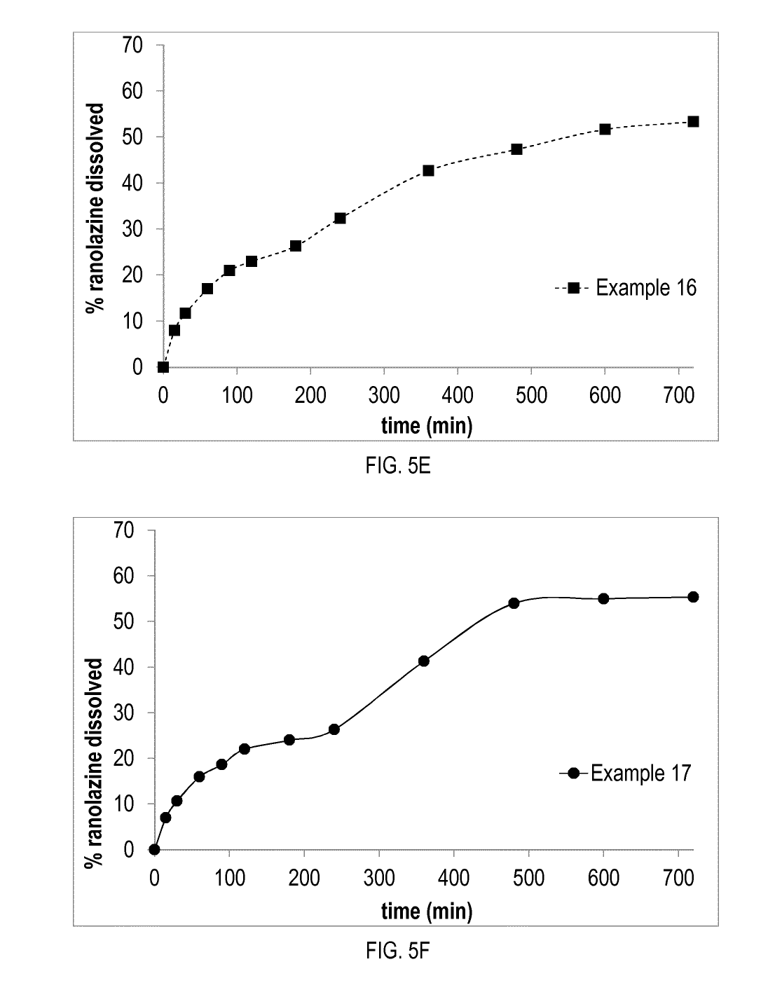

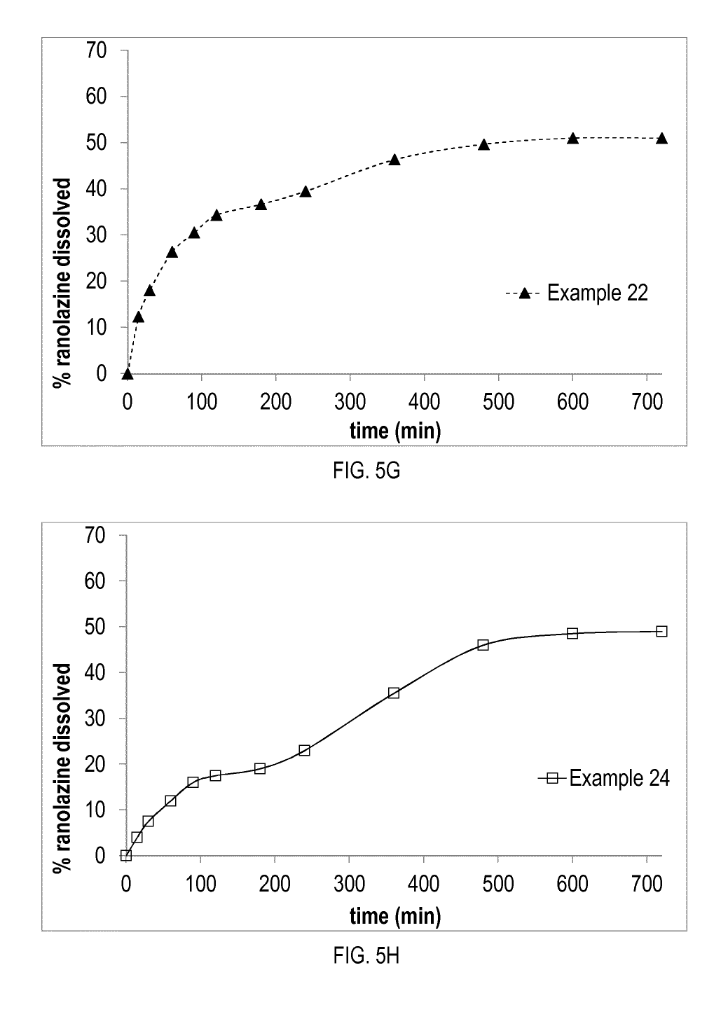

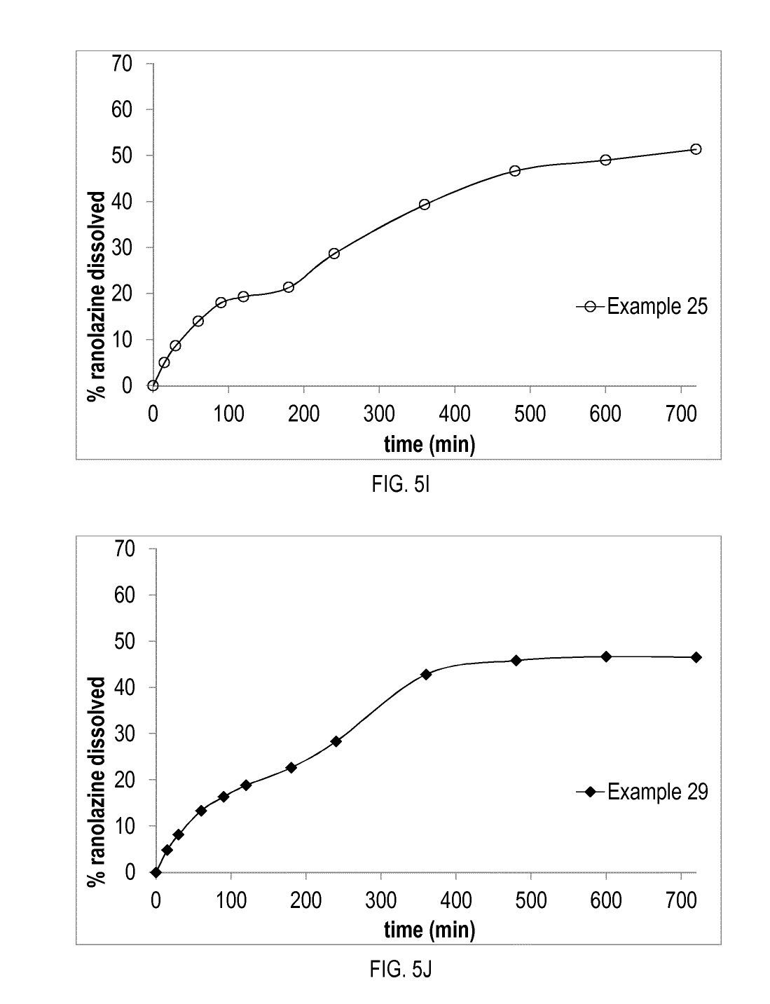

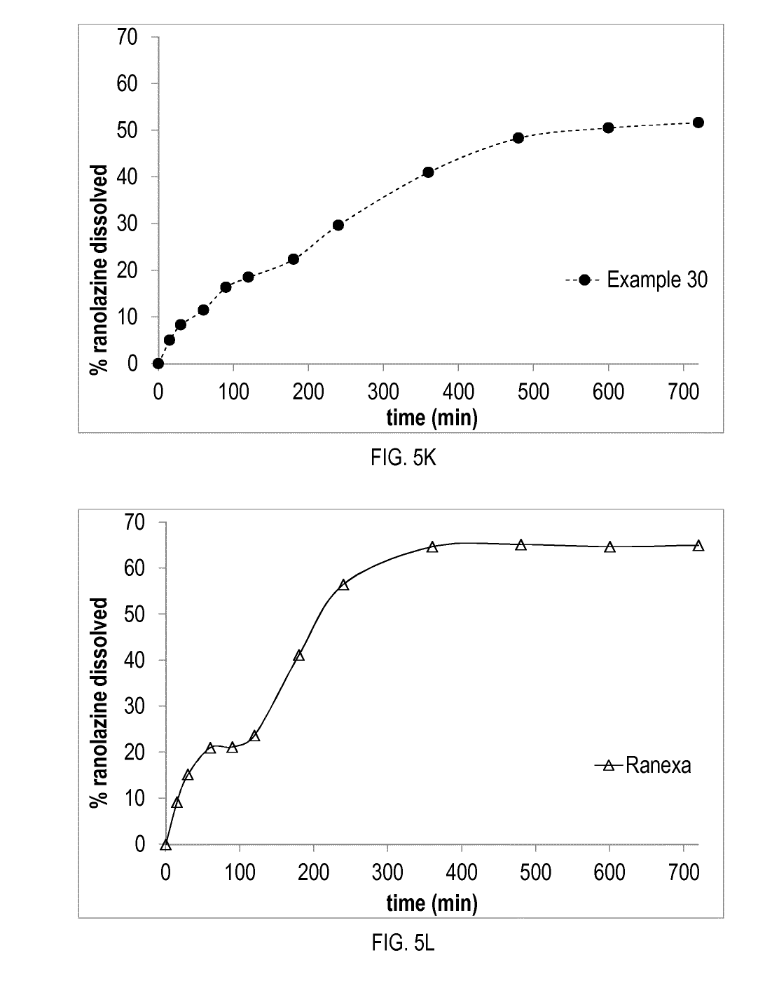

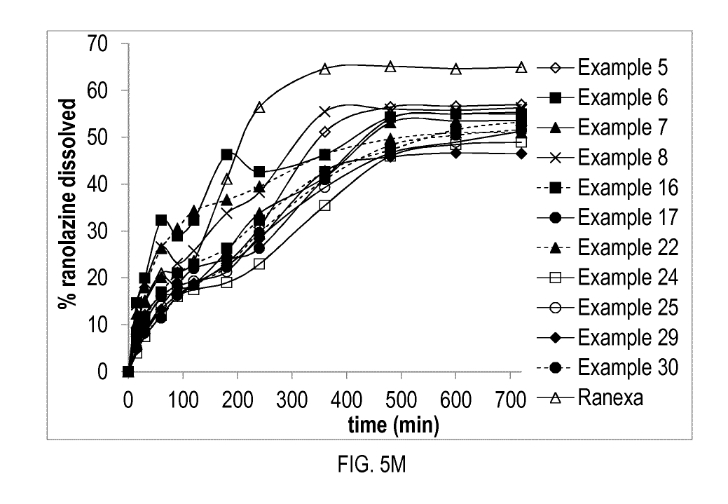

Tablets can have different shapes, which are determined by the shapes of the die and punches. In FIG. 4 different shapes for the bases of the tablets are depicted, but any other shape may be suitable for the present invention. Some embodiments of the invention are depicted in FIG. 3, for such embodiments round shape for the tablet bases has been selected, but any other form (among the ones of FIG. 4 or any other) may be used within the scope of the invention.

The bases and lateral surface of the embodiments in FIG. 3 are flat, but may have different form such as curved, convex, concave, wavy or any other or combinations thereof.

It is known by the skilled in the art that the dissolution of a drug in a tablet depends on the surface to volume ratio and that shapes with a higher surface to volume ratio tend to increase the dissolution rate, while shapes with lower surface to volume ratio tend to decrease it. For instance, an oblong tablet has a higher surface to volume ratio than a round tablet. Depending of the desired dissolution rate one specific tablet shape would be preferred.

pH independent retardant polymers are commonly gel-forming polymers with a viscosity typically ranging from 20,000 to 300,000 mPa·s measured in a 2% (w/v) aqueous solution of the pH independent retardant polymers using the method for the determination of viscosity of the European Pharmacopeia 8th edition 2015 (8.5). In a preferred embodiment of the invention, the viscosity ranges from 30,000 to 150,000 mPa·s measured using the same method. In another embodiment the viscosity ranges from 75,000 to 140,000 mPa·s measured using the same method.

Embodiment 1 is a multiple compressed tablet obtainable by a process comprising at least two compression cycles, wherein
in each of the compression cycles a pharmaceutical composition, comprising one or more pharmaceutically acceptable excipients, is used,
at least one of such pharmaceutical compositions comprises one or more release retardant agents,
at least two of such pharmaceutical compositions comprise ranolazine and have a different quantitative and/or qualitative composition.

Embodiment 2 is the multiple compressed tablet according to embodiment 1, wherein at least one release retardant agent is a pH independent release retardant agent.

Embodiment 3 is the multiple compressed tablet according to embodiment 2, wherein all of the release retardant agents are pH independent release retardant agents.

Embodiment 4 is the multiple compressed tablet according to any of the preceding embodiments, which is substantially free from pH dependent release retardant agents.

Embodiment 5 is the multiple compressed tablet according to any of the preceding embodiments, wherein the release retardant agent is a pH independent release retardant polymer.

Embodiment 6 is the multiple compressed tablet according to embodiment 5, wherein the pH independent release retardant polymer is selected from: hydroxypropyl methylcellulose, hydroxypropyl cellulose, methylcellulose, polyvinylpyrrolidone and neutral poly(meth)acrylate esters.

Embodiment 7 is the multiple compressed tablet according to embodiment 6, wherein the pH independent release retardant polymer is hydroxypropyl methylcellulose.

Embodiment 8 is the multiple compressed tablet according to embodiments 5, 6 or 7, wherein the pH independent release retardant polymer has a viscosity between 20,000 and 300,000 mPa·s at 20° C. measured in a 2% (w/v) aqueous solution of the pH independent release retardant polymer.

Embodiment 9 is the multiple compressed tablet according to embodiment 8, wherein the pH independent release retardant polymer has a viscosity between 30,000 and 150,000 mPa·s at 20° C. measured in a 2% (w/v) aqueous solution of the pH independent release retardant polymer.

Embodiment 10 is the multiple compressed tablet according to embodiment 9, wherein the pH independent release retardant polymer has a viscosity between 75,000 and 140,000 mPa·s at 20° C. measured in a 2% (w/v) aqueous solution of the pH independent release retardant polymer.

Embodiment 11 is the multiple compressed tablet according to any of the preceding embodiments, wherein all the pharmaceutical compositions which comprise ranolazine used in the compression cycles comprises at least one release retardant agent.

Embodiment 12 is the multiple compressed tablet according to any of the preceding embodiments, wherein ranolazine is the only drug.

Embodiment 13 is the multiple compressed tablet according to any of the preceding embodiments, wherein the total ranolazine content is higher than 50% with respect to the total weight of the tablet.

Embodiment 14 is the multiple compressed tablet according to any of the preceding embodiments, wherein the total ranolazine content is higher than 65% with respect to the total weight of the tablet.

Embodiment 15 is the multiple compressed tablet according to any of the preceding embodiments, wherein the total ranolazine content is higher than 70% with respect to the total weight of the tablet.

Embodiment 16 is the multiple compressed tablet according to any of the preceding embodiments, wherein at least one of the pharmaceutical compositions used in the compression cycles is used in more than one compression cycle.

Embodiment 17 is the multiple compressed tablet according to embodiment 16, in which only two different pharmaceutical compositions are used in the compression cycles.

Embodiment 18 is the multiple compressed tablet according to any of the previous embodiments which is a bilayer tablet, a trilayer tablet, a tablet into tablet, an inlay tablet or a multilayer tablet.

Embodiment 19 is the multiple compressed tablet according to embodiment 18, which is a bilayer tablet.

Embodiment 20 is the multiple compressed tablet according to embodiment 18, which is a trilayer tablet.

Embodiment 21 is the trilayer tablet according to embodiment 20, wherein the two outer layers are obtained using the same pharmaceutical composition

Embodiment 22 is the trilayer tablet according to embodiments 20 or 21, wherein in the preparation of the two outer layers, substantially the same amount of a pharmaceutical composition is used in the two layers.

Embodiment 23 is the multiple compressed tablet according to embodiment 18, which is a press-coated tablet.

Embodiment 24 is the press-coated tablet according to embodiment 23, wherein in the compression cycles used to prepare the two outer parts, the same pharmaceutical composition is used in each compression cycle.

Embodiment 25 is the press-coated tablet according to embodiments 23 or 24, wherein in the compression cycles used to prepare the two outer parts, substantially the same amount of a pharmaceutical composition is used in each compression cycle.

Embodiment 26 is the multiple compressed tablet according to embodiment 18, which is an inlay tablet.

Embodiment 27 is the multiple compressed tablet according to embodiment 18, which is a multilayer tablet.

Embodiment 28 is the multiple compressed tablet according to any of embodiments 18 to 27 wherein at least one of the outer pharmaceutical compositions comprises at least one release retardant agent.

Embodiment 29 is the trilayer tablet according to embodiment 28 wherein the outer pharmaceutical composition or compositions comprises at least one release retardant agent.

Embodiment 30 is the press-coated tablet according embodiment 28 wherein the outer pharmaceutical composition comprises at least one release retardant agent.

Embodiment 31 is the inlay tablet according to embodiment 28 wherein the outer pharmaceutical composition comprises at least one release retardant agent.

Embodiment 32 is the multiple compressed tablet according to any of the embodiments 28, 29, 30 or 31, wherein the inner or intermediate pharmaceutical composition comprises less than 50% of the total ranolazine content.

Embodiment 33 is the multiple compressed tablet according to any of embodiments 30 or 31, wherein the inner pharmaceutical composition is film-coated or sugar coated.

Embodiment 34 is the multiple compressed tablet according to any of the preceding embodiments, wherein the tablet is film-coated or sugar coated.

Embodiment 35 is a multiple compressed tablet according to any of the preceding embodiments for use in the treatment of angina pectoris.

Embodiment 36 is a process for the preparation of the multiple compressed tablet according to any of the preceding embodiments comprising:
a) independently mixing all the components of all the pharmaceutical compositions,
b) optionally tableting one or more pharmaceutical compositions,
c) either
   i. charging and optionally precompressing a pharmaceutical composition different from the one used in the previous cycle on the result of the previous cycle, or
   ii. placing a tablet of step b), prepared using a pharmaceutical composition different from the one used in the previous cycle, on the result of the previous cycle,
d) repeating step c) at least one time,
e) compressing the result of the previous step.

Embodiment 37 is the process according to embodiment 36 for the preparation of a bilayer tablet according to embodiment 19, comprising:
a) independently mixing all the components of two pharmaceutical compositions,
b) charging and at least precompressing one pharmaceutical composition in the die of a tabletting machine,
c) charging the other pharmaceutical composition on the result of step b), and
d) compressing the result of step c).

Embodiment 38 is the process according to embodiment 36 for the preparation of trilayer tablets according to any of the embodiments 20 to 22 comprising:
a) independently mixing all the components of all the pharmaceutical compositions,
b) charging and at least precompressing one pharmaceutical composition in the die of a tabletting machine,
c) charging and at least precompressing another pharmaceutical composition on the result of step b),
d) charging
   i. the pharmaceutical composition used in step b), or
   ii. a pharmaceutical composition different from the one used in steps b) and c) on the result of step c), and
e) compressing the result of step d).

Embodiment 39 is the process according to embodiment 36 for the preparation of a press-coated tablet according to any of the embodiments 23 to 25 comprising:
a) independently mixing all the components of all the pharmaceutical compositions,
b) tableting one pharmaceutical composition,
c) charging and at least precompressing another pharmaceutical composition in the die of a tabletting machine,
d) placing the tablet of step b) on the result of step c),
e) charging
   i. the pharmaceutical composition used in step c), or
   ii. a pharmaceutical composition different from the one used in steps b) or c) on the result of step d), and
f) compressing the result of step e).

Embodiment 40 is the process according to embodiment 36 for the preparation of an inlay tablet according to embodiment 26 comprising:
a) independently mixing all the components of two pharmaceutical compositions,
b) tableting one pharmaceutical composition,
c) charging and at least precompressing the other pharmaceutical composition in the die of a tabletting machine,
d) placing the tablet of step b) on the result of step c), and
e) compressing the result of step d).

Embodiment 41 is the process according to embodiment 36 for the preparation of an inlay tablet according to embodiment 26 comprising:
a) independently mixing all the components of two pharmaceutical compositions,
b) tableting one pharmaceutical composition,
c) placing the tablet of step b) on the die of a tabletting machine,
d) charging the other pharmaceutical composition in the result of step c), and
e) compressing the result of step d).

Embodiment 42 is the process according to embodiment 36 for the preparation of a multilayer tablet according to embodiment 27 comprising:
a) independently mixing all the components of all the pharmaceutical compositions,
b) charging and at least precompressing one pharmaceutical composition in the die of a tabletting machine,
c) charging and at least precompressing a pharmaceutical composition different from that used in the previous cycle on the result of the previous cycle,
d) repeating step c) as many times as desired, and
e) compressing the result of step d).

Embodiment 43 is the process according to any of the embodiments 36 to 42 further comprising a film coating or a sugar coating step.

The following examples are illustrative and are not considered to limit the scope of the invention.

### EXAMPLES

### Manufacturing Process Examples

Manufacturing of the Inner and Outer Pharmaceutical Compositions

All the components of the inner or the outer pharmaceutical compositions except for the glidant (in these examples, hydrophilic fumed silica), the lubricant (in these examples, the amount of sodium stearyl fumarate listed below water) and the optional external part of the binder (in these examples, the amount of hypromellose K100 LVCR listed below water in the table) are mixed and granulated with binder aqueous solution in a high shear mixer granulator. The resulting granules are dried in a fluid bed dryer and the glidant, the lubricant and the optional external part of the binder, if present, are added to the mixture of granules and mixed in a blender to obtain the inner or the outer fraction.

Alternatively the inner and/or the outer pharmaceutical compositions can be prepared using compactation, dry granulation or can be compressed directly.

### Example M1 (Bilayer)

The desired amount of the inner pharmaceutical compositions is charged inside of the die of a bi-layer rotary tabletting machine and precompressed with a force of 1.0 kN. Then the desired amount of the outer pharmaceutical compositions is charged inside of the die on the previous composition, precompressed with a force of 1.0 kN and finally compressed with a force of 18 kN.

### Example M2 (Trilayer)

Part of the desired amount of the outer pharmaceutical compositions is charged inside of the die of a three-layer rotary tabletting machine and precompressed with a force of 0.5 kN. Then the desired amount of the intermediate pharmaceutical compositions is charged inside of the die on the previous composition and precompressed with a force of 1.0 kN. Finally the remaining part of the outer pharmaceutical compositions is charged inside of the die on the previous composition and compressed with a force of 18 kN.

In the present example, the amount of the outer pharmaceutical composition is divided in two equal parts and used to prepare the first and third layer in the tablet.

### Example M3 (Multilayer Tablets)

Part of the desired amount of the outer pharmaceutical compositions is charged inside of the die of a multi-layer rotary tabletting machine and precompressed with a force of 0.5 kN. Then part of the desired amount of the inner pharmaceutical compositions is charged inside of the die and precompressed with a force of 1.0 kN on the previous composition. The process is repeated as many times as layers desired. Finally the mixture is compressed with a force of 18 kN.

In the specific examples that refer to Example M3 5-layer tablets are prepared with the following pattern: outer, intermediate, outer, intermediate and outer pharmaceutical compositions. Only one intermediate and one outer pharmaceutical composition were used in the present examples.

### Example M4 (Press Coated Tablet)

The desired amount of the inner pharmaceutical compositions is tabletted in the desired manner.

Part of the desired amount of the outer pharmaceutical compositions is charged inside of the die of a rotary tabletting machine and precompressed with a force of 0.5 kN. The inner tablet is placed partially sunken in the previously placed outer pharmaceutical compositions and precompressed with a force of 0.5 kN. Then the rest of the outer pharmaceutical compositions is charged inside of the die and then compressed with a force from 18 kN.

In the present example the amount of the outer pharmaceutical composition is divided in two equal parts that are charged in the die in the two compression cycles wherein an outer pharmaceutical composition is charged.

### Example M5 (Inlay Tablet)

The desired amount of the inner pharmaceutical compositions is tabletted in the desired manner.

The desired amount of the outer pharmaceutical compositions is charged inside of the die of a rotary tabletting machine and precompressed with a force of 0.5 kN. The inner tablet is partially sunken in the previously placed outer pharmaceutical compositions and precompressed with a force of 0.5 kN. Finally the mixture is compressed with a force of 18 kN.

In any of the Examples M1 to M5, the tablets obtained might optionally be film or sugar coated.

In the following examples, inner, intermediate and outer pharmaceutical compositions have been obtained by wet granulation as described above, except for the cases wherein no water is indicated in the quantitative composition. In these later cases mixing and direct compression was carried out. Where the compositions include a coating (such as Opadry pink or Opadry AMB white), this coating has been applied to the tablet by a conventional coating process using the amounts of water specified in the following table.

### Quantitative Outer Pharmaceutical Compositions Examples:

| Ingredient | O1 (%) | O2 (%) | O3 (%) | O4 (%) | O5 (%) | O6 (%) | O7 (%) | O8 (%) | O9 (%) |
|---|---|---|---|---|---|---|---|---|---|
| Ranolazine | 73.10 | 73.17 | 71.43 | 71.43 | 71.43 | 71.43 | 71.43 | 71.43 | 71.43 |
| Hypromellose K100M Premium | 6.14 | 6.10 | 5.94 | 5.94 | 5.94 | 7.14 | 4.02 | 7.00 | 5.00 |
| Hypromellose K100 LVCR | 6.14 | 6.10 | 5.94 | 5.94 | 5.94 | 7.14 | 4.02 | 7.00 | 5.00 |
| Hypromellose 2910 E5 | — | — | 2.38 | 2.38 | 2.38 | 2.38 | 2.38 | 2.38 | 2.38 |
| Carmellose Sodium | 10.23 | 10.24 | 10.03 | 10.03 | 10.03 | 7.62 | 13.87 | 7.90 | 11.90 |
| Water* | 0.33 | 0.33 | 0.63 | 0.44 | 0.48 | 0.48 | 0.48 | 0.35 | 0.31 |
| Aerosil 200** | 1.46 | 1.46 | 1.43 | 1.43 | 1.43 | 1.43 | 1.43 | 1.43 | 1.43 |
| Sodium stearyl fumarate | 2.92 | 2.93 | 2.86 | 2.86 | 2.86 | 2.86 | 2.86 | 2.86 | 2.86 |
| Total | 100.00 | 100.00 | 100.00 | 100.00 | 100.00 | 100.00 | 100.00 | 100.00 | 100.00 |

*mg(water)/mg(ranolazine);
**Hydrophilic fumed silica with specific surface area of 200 m²/g.

### Quantitative Inner or Intermediate Pharmaceutical Compositions Examples:

| Ingredient | I1 (%) | I2 (%) | I3 (%) | I4 (%) | I5 (%) | I6 (%) | I7 (%) |
|---|---|---|---|---|---|---|---|
| Ranolazine | 69.69 | 69.69 | 69.69 | 69.69 | 69.69 | 69.69 | — |
| Microcrystalline cellulose 102 | 14.29 | 14.29 | 12.29 | 12.29 | 11.29 | 12.29 | 85.00 |
| Hypromellose K100M Premium | — | — | 2.00 | 2.00 | 3.00 | 2.00 | — |
| Sodium starch glycolate | 2.44 | 2.44 | 2.44 | 2.44 | 2.44 | 2.44 | 14.00 |
| polyvinylpyrrolidone K25 | 3.83 | — | — | — | — | — | — |
| polyvinylpyrrolidone K30 | 0.00 | 3.83 | 3.83 | 3.83 | 3.83 | 3.83 | — |
| Water* | 0.50 | 0.50 | 0.50 | 0.40 | 0.40 | 0.50 | — |
| Hypromellose K100 LVCR | 6.97 | 6.97 | 6.97 | 6.97 | 6.97 | 6.97 | — |
| Aerosil 200** | 0.93 | 0.93 | 0.92 | 0.92 | 0.92 | 0.92 | — |
| Sodium stearyl fumarate | 1.86 | 1.86 | 1.86 | 1.86 | 1.86 | 1.86 | 1.00 |
| Total | 100.00 | 100.00 | 100.00 | 100.00 | 100.00 | 100.00 | 100.00 |

*mg(water)/mg(ranolazine);
**Hydrophilic fumed silica with specific surface area of 200 m²/g.

Composition examples (amounts in mg/tablet):

| | Outer pharmaceutical compositions | | | | Inner/intermediate pharmaceutical compositions | | | | manufacturing | opadry | opadry AMB | |
|---|---|---|---|---|---|---|---|---|---|---|---|---|
| Example | amount | example | water* | ranolazine | amount | example | water* | ranolazine | process | pink | white | water** |
| 1 | 499.80 | O5 | 170.65 | 357.00 | 205.20 | I2 | 715.00 | 143.00 | M2 | | | |
| 2 | 630.00 | O5 | 215.10 | 450.00 | 71.75 | I2 | 250.00 | 50.00 | M2 | | | |
| 3 | 472.50 | O5 | 161.25 | 337.50 | 53.81 | I2 | 18.75 | 37.50 | M2 | | | |
| 4 | 420.00 | O5 | 143.40 | 300.00 | 287.00 | I2 | 100.00 | 200.00 | M2 | | | |
| 5 | 1260.00 | O5 | 430.20 | 900.00 | 143.00 | I2 | 50.00 | 100.00 | M5 | | | |
| 6 | 840.00 | O5 | 286.80 | 600.00 | 574.00 | I2 | 200.00 | 400.00 | M2 | | | |
| 7 | 1120.00 | O5 | 382.40 | 800.00 | 287.00 | I2 | 100.00 | 200.00 | M2 | | | |
| 8 | 910.00 | O5 | 310.70 | 650.00 | 502.25 | I2 | 175.00 | 350.00 | M2 | | | |
| 9 | 1260.00 | O5 | 430.20 | 900.00 | 143.00 | I2 | 50.00 | 100.00 | M2 | | | |
| 10 | 1120.00 | O5 | 382.40 | 800.00 | 287.00 | I2 | 100.00 | 200.00 | M2 | 42.00 | | 238.00 |
| 11 | 840.00 | O5 | 286.80 | 600.00 | 574.00 | I2 | 200.00 | 400.00 | M2 | | 141.00 | 800.00 |
| 12 | 1120.00 | O5 | 382.40 | 800.00 | 287.00 | I2 | 100.00 | 200.00 | M2 | 42.00 | | 238.00 |
| 13 | 840.00 | O5 | 286.80 | 600.00 | 574.00 | I2 | 200.00 | 400.00 | M2 | | 141.00 | 800.00 |
| 14 | 840.00 | O5 | 286.80 | 600.00 | 215.25 | I2 | 75.00 | 150.00 | M4 | | | |
| 15 | 1400.00 | O5 | 478.00 | 1000.00 | 200.00 | I7 | 0.00 | 0.00 | M2 | | | |
| 16 | 1120.00 | O5 | 382.40 | 800.00 | 287.00 | I3 | 100.00 | 200.00 | M2 | | | |
| 17 | 1120.00 | O5 | 382.40 | 800.00 | 287.00 | I2 | 100.00 | 200.00 | M4 | | | |
| 18 | 840.00 | O5 | 286.80 | 600.00 | 215.25 | I2 | 75.00 | 150.00 | M2 | | | |
| 19 | 1120.00 | O5 | 382.40 | 800.00 | 287.00 | I2 | 100.00 | 200.00 | M2 | | | |
| 20 | 840.00 | O5 | 286.80 | 600.00 | 215.25 | I2 | 75.00 | 150.00 | M2 | 31.50 | | 238.00 |
| 21 | 1120.00 | O7 | 382.40 | 800.00 | 287.00 | I2 | 100.00 | 200.00 | M2 | | | |
| 22 | 1400.00 | O6 | 478.00 | 1000.00 | 200.00 | I7 | 0.00 | 0.00 | M2 | | | |
| 23 | 1120.00 | O7 | 382.40 | 800.00 | 287.00 | I3 | 100.00 | 200.00 | M2 | | | |
| 24 | 1120.00 | O5 | 382.40 | 800.00 | 287.00 | I2 | 100.00 | 200.00 | M4 | 42.00 | | 238.00 |
| 25 | 1120.00 | O5 | 382.40 | 800.00 | 287.00 | I5 | 80.00 | 200.00 | M2 | 42.00 | | 238.00 |
| 28 | 1120.00 | O8 | 300.00 | 800.00 | 287.00 | I3 | 100.00 | 200.00 | M2 | 42.00 | | 238.00 |
| 27 | 1120.00 | O7 | 382.40 | 800.00 | 287.00 | I2 | 100.00 | 200.00 | M4 | 42.00 | | 238.00 |
| 28 | 1120.00 | O9 | 250.40 | 800.00 | 287.00 | I2 | 100.00 | 200.00 | M4 | 42.00 | | 239.00 |
| 29 | 1120.00 | O9 | 250.40 | 800.00 | 287.00 | I2 | 100.00 | 200.00 | M4 | 43.00 | | 240.00 |
| 30 | 1120.00 | O5 | 382.40 | 800.00 | 287.00 | I5 | 80.00 | 200.00 | M2 | 44.00 | | 241.00 |
| 31 | 472.49 | O3 | 212.63 | 337.50 | 53.81 | I4 | 15.00 | 37.50 | M5 | | | |
| 32 | 307.50 | O2 | 74.25 | 225.00 | 215.24 | I6 | 75.00 | 150.00 | M1 | | | |
| 33 | 839.98 | O4 | 264.00 | 600.00 | 573.97 | I4 | 160.00 | 400.00 | M3 | | | |
| 34 | 478.80 | O1 | 115.50 | 350.00 | 215.24 | I6 | 75.00 | 150.00 | M3 | | | |
| 35 | 1119.98 | O4 | 352.00 | 800.00 | 286.99 | I1 | 100.00 | 200.00 | M1 | | | |
| 36 | 922.51 | O2 | 222.75 | 675.00 | 107.62 | I6 | 37.50 | 75.00 | M3 | | | |
| 37 | 629.99 | O3 | 283.50 | 450.00 | 71.75 | I6 | 25.00 | 50.00 | M1 | | | |
| 38 | 314.99 | O4 | 99.00 | 225.00 | 215.24 | I1 | 75.00 | 150.00 | M3 | | | |
| 39 | 307.50 | O2 | 74.25 | 225.00 | 215.24 | I6 | 75.00 | 150.00 | M5 | | | |
| 40 | 559.99 | O4 | 176.00 | 400.00 | 143.49 | I6 | 50.00 | 100.00 | M1 | | | |
| 41 | 615.01 | O2 | 148.50 | 450.00 | 71.75 | I1 | 25.00 | 50.00 | M3 | | | |
| 42 | 615.60 | O1 | 148.50 | 450.00 | 430.48 | I6 | 150.00 | 300.00 | M3 | | | |
| 43 | 944.98 | O3 | 425.25 | 675.00 | 107.62 | I6 | 37.50 | 75.00 | M1 | | | |
| 44 | 419.99 | O4 | 132.00 | 300.00 | 286.99 | I1 | 100.00 | 200.00 | M5 | | | |
| 45 | 839.98 | O4 | 264.00 | 600.00 | 215.24 | I4 | 60.00 | 150.00 | M1 | | | |
| 46 | 629.99 | O3 | 283.50 | 450.00 | 430.48 | I1 | 150.00 | 300.00 | M5 | | | |
| 47 | 944.98 | O3 | 425.25 | 675.00 | 107.62 | I6 | 37.50 | 75.00 | M1 | | | |
| 48 | 820.01 | O2 | 198.00 | 600.00 | 573.97 | I1 | 200.00 | 400.00 | M5 | | | |
| 49 | 307.50 | O2 | 74.25 | 225.00 | 215.24 | I4 | 60.00 | 150.00 | M5 | | | |
| 50 | 615.01 | O2 | 148.50 | 450.00 | 71.75 | I4 | 20.00 | 50.00 | M5 | | | |
| 51 | 957.59 | O1 | 231.00 | 700.00 | 430.48 | I4 | 120.00 | 300.00 | M1 | | | |
| 52 | 478.80 | O1 | 115.50 | 350.00 | 215.24 | I6 | 75.00 | 150.00 | M5 | | | |
| 53 | 472.49 | O3 | 212.63 | 337.50 | 53.81 | I1 | 18.75 | 37.50 | M3 | | | |
| 54 | 629.99 | O3 | 283.50 | 450.00 | 71.75 | I6 | 25.00 | 50.00 | M3 | | | |
| 55 | 839.98 | O3 | 378.00 | 600.00 | 573.97 | I6 | 200.00 | 400.00 | M3 | | | |
| 56 | 956.68 | O2 | 231.00 | 700.00 | 430.48 | I1 | 150.00 | 300.00 | M1 | | | |
| 57 | 820.01 | O2 | 198.00 | 600.00 | 215.24 | I6 | 75.00 | 150.00 | M1 | | | |
| 58 | 419.99 | O3 | 189.00 | 300.00 | 286.99 | I1 | 100.00 | 200.00 | M5 | | | |
| 59 | 957.59 | O1 | 231.00 | 700.00 | 430.48 | I4 | 120.00 | 300.00 | M1 | | | |
| 60 | 734.99 | O3 | 330.75 | 525.00 | 322.86 | I6 | 112.50 | 225.00 | M5 | | | |

*mg of water per tablet used in the granulation step.
**mg of water per tablet used in the film coating step. All the water is removed during the process.

1.-15. (canceled)

16. A multiple compressed tablet comprising:
one or more pharmaceutical compositions,
wherein the multiple compressed tabled is obtained by a process comprising at least two compression cycles, wherein
in each of the compression cycles, one or more pharmaceutical compositions, comprising one or more pharmaceutically acceptable excipients is used,
at least one of said pharmaceutical compositions comprises one or more release retardant agents, and
at least two of said pharmaceutical compositions comprise ranolazine and have a different quantitative and/or qualitative composition.

17. The multiple compressed tablet according to claim 16, wherein at least one release retardant agent is a pH independent release retardant agent.

18. The multiple compressed tablet according to claim 17, wherein the pH independent release retardant polymer is selected from: hydroxypropyl methylcellulose, hydroxypropyl cellulose, methylcellulose, polyvinylpyrrolidone, neutral poly(meth)acrylate esters and mixtures thereof.

19. The multiple compressed tablet according to claim 18, wherein the pH independent release retardant polymer is hydroxypropyl methylcellulose.

20. The multiple compressed tablet according to claim 18, wherein the pH independent release retardant polymer has a viscosity between 75,000 and 140,000 mPa·s at 20° C. measured in a 2% (w/v) aqueous solution of the pH independent release retardant polymer.

21. The multiple compressed tablet according to claim 16, wherein the total ranolazine content is higher than 70% with respect to the total weight of the tablet.

22. The multiple compressed tablet according to claim 16, in which only two different pharmaceutical compositions are used in the compression cycles.

23. The multiple compressed tablet according to claim 16, which is a bilayer tablet, a trilayer tablet, a tablet into tablet, an inlay tablet or a multilayer tablet.

24. The multiple compressed tablet according to claim 23, which is a bilayer tablet.

25. The multiple compressed tablet according to claim 23, which is a press-coated tablet.

26. The multiple compressed tablet according to claim 23 wherein at least one of the outer pharmaceutical compositions comprises at least one release retardant agent.

27. The multiple compressed tablet according to claim 16, wherein the inner or intermediate pharmaceutical composition comprises less than 50% of the total ranolazine content.

28. A method for treating angina pectoris to a person in need thereof, comprising:
administering a pharmaceutically effective amount of the multiple compressed tablet according to claim 16 a person in need thereof.

29. A process for the preparation of the multiple compressed tablet according to claim 16 comprising:
a) independently mixing all components of all the pharmaceutical compositions,
b) optionally tableting one or more pharmaceutical compositions,
c) either
i. charging and optionally precompressing a pharmaceutical composition different from the one used in the previous cycle on the result of the previous cycle, or
ii. placing a tablet of step b), prepared using a pharmaceutical composition different from the one used in the previous cycle, on the result of the previous cycle,
d) repeating step c) at least one time,
e) compressing the result of the previous step.
